(19) United States
(12) Patent Application Publication
(10) Pub. No.: US 2023/0094202 A1
(43) Pub. Date: Mar. 30, 2023

(54) ENDOSCOPE

(71) Applicant: FUJIFILM Corporation, Tokyo (JP)

(72) Inventor: Wataru INOUE, Kanagawa (JP)

(73) Assignee: FUJIFILM Corporation, Tokyo (JP)

(21) Appl. No.: 17/930,975

(22) Filed: Sep. 9, 2022

(30) Foreign Application Priority Data
Sep. 30, 2021 (JP) 2021-161244
Feb. 25, 2022 (JP) 2022-027848

(51) Int. Cl. A61B 1/00

(52) U.S. Cl. CPC A61B 1/00096; A61B 1/0011; A61B 1/05

(57) ABSTRACT

There is provided an endoscope of which a structure is simple and co-rotation of an external cable can be prevented.

A fixing member to be rotated integrally with a knob in a direction around an axis is disposed in an operation unit body, and a part of a light guide in a longitudinal direction is fixed by a fixing part of the fixing member. A light guide-insertion space is formed between the fixing part and a distal end-side opening portion in the operation unit body. A light guide is inserted into and disposed in the light guide-insertion space so that tension is not applied to the light guide present between the fixing part and the distal end-side opening portion even in a case where the fixing member is rotated in the direction around the axis by the knob.

### CROSS-REFERENCE TO RELATED APPLICATION

The present application claims priorities under 35 U.S.C. § 119(a) to Japanese Patent Application No. 2021-161244 filed on Sep. 30, 2021 and Japanese Patent Application No. 2022-027848 filed on Feb. 25, 2022, each of which is hereby expressly incorporated by reference, in its entirety, into the present application.

### BACKGROUND OF THE INVENTION

### 1. Field of the Invention

The present invention relates to an endoscope including an insertion unit.

### 2. Description of the Related Art

A rigid endoscope is known as an endoscope used for endoscopic surgery or the like. Further, an oblique-viewing endoscope of which a diagonal front side with respect to an insertion axis of an insertion unit corresponds to a visual field direction (observation direction) is known as this rigid endoscope. The oblique-viewing endoscope comprises an insertion unit that is to be inserted into an object to be examined and an operation unit body that is connected to a proximal end side of the insertion unit. JP2021-510103A and U.S. Pat. No. 5,621,830A disclose such oblique-viewing endoscopes of which visual field directions can be changed.

The oblique-viewing endoscope disclosed in JP2021-510103A includes a shaft (insertion unit), a handle (operation unit body), and a rotary swivel. An objective lens is disposed at a distal end of the shaft. In a case where a visual field direction is to be changed, a user holds the rotary swivel and rotates the shaft in a direction around an axis using the handle. Accordingly, the visual field direction of the objective lens is rotated about the axis of the shaft.

The oblique-viewing endoscope disclosed in U.S. Pat. No. 5,621,830A includes an insertion unit, an operation unit body, and a rotational operation ring. A distal end optical system is disposed on a distal end side of the insertion unit. In a case where a visual field direction is to be changed, a user rotationally operates the rotational operation ring provided on the operation unit body to rotate the insertion unit in a direction around an axis. Accordingly, the visual field direction of the distal end optical system is rotated about the axis of the insertion unit.

### SUMMARY OF THE INVENTION

In the oblique-viewing endoscope disclosed in JP2021-510103A, a user should rotationally operate the operation unit body while holding the rotary swivel provided on the distal end side of the operation unit body in order to rotate an outer pipe of the insertion unit in the direction around the axis together with a distal end optical system (objective lens) as an operation for changing a visual field direction. For this reason, there is a problem in that it is difficult to operate the oblique-viewing endoscope.

Meanwhile, a configuration in which an outer pipe of the insertion unit is rotated in the direction around the axis together with the distal end optical system in a case where a user rotationally operates the rotational operation ring provided on the operation unit body is employed in the oblique-viewing endoscope disclosed in U.S. Pat. No. 5,621,830A. According to this configuration, there is an advantage in that an operation for changing a visual field direction is easy as compared to the oblique-viewing endoscope disclosed in JP2021-510103A.

A configuration in which distal end-side optical fibers and proximal end-side optical fibers are connected to each other using a rotary joint so as to be rotatable relative to each other is employed in the oblique-viewing endoscope disclosed in U.S. Pat. No. 5,621,830A in order to prevent torsion (co-rotation) of an optical fiber and an external cable in a case where the outer pipe is rotated. However, this configuration has a problem in that a structure is likely to be complicated since the distal end-side optical fibers and the proximal end-side optical fibers should be evenly arranged in a ring shape in the operation unit body. As a result, the operation unit body is caused to increase in size, which is a factor of reducing operability.

Accordingly, it is desired to develop an oblique-viewing endoscope of which the structure is simple and the co-rotation of an external cable can be prevented.

The present invention has been made in consideration of such circumstances, and an object of the present invention is to provide an endoscope of which the structure is simple and the co-rotation of an external cable can be prevented.

In order to achieve the object of the present invention, an endoscope according to an aspect of the present invention comprises: an outer pipe that forms an insertion unit; a pipe-like operation unit body that is connected to a proximal end side of the outer pipe and that supports the outer pipe to allow the outer pipe to be rotatable in a direction around an axis of the insertion unit; an annular rotational operation member that is fixed to the proximal end side of the outer pipe and that rotates the outer pipe in the direction around the axis with respect to the operation unit body; a protection sheath that is inserted into the outer pipe and that is rotatable integrally with the outer pipe; a distal end optical system that is provided on a distal end side of the protection sheath; a flexible light guide that is disposed in a space between the outer pipe and the protection sheath and that includes a light emitting end on a distal end side of the outer pipe; an inner sheath that is inserted into the protection sheath and that is rotatable relative to the protection sheath in the direction around the axis; an image pickup unit that is provided on a distal end side of the inner sheath and that picks up an image of light passing through the distal end optical system; an external cable which is connected to a proximal end side of the operation unit body and into which the light guide is inserted from a distal end-side opening portion; and a fixing member that is disposed in the operation unit body, includes a fixing part for fixing a part of the light guide in a longitudinal direction, and is rotatable integrally with the rotational operation member in the direction around the axis. A light guide-insertion space is formed between the fixing part and the distal end-side opening portion in the operation unit body. In a case where the fixing member is rotated in the direction around the axis by the rotational operation member, the light guide is inserted into and disposed in the light guide-insertion space in a state where tension is not applied to the light guide present between the fixing part and the distal end-side opening portion.

In order to achieve the object of the present invention, an endoscope according to another aspect of the present invention comprises: an outer pipe that forms an insertion unit; a pipe-like operation unit body that is connected to a proximal end side of the outer pipe and that supports the outer pipe to allow the outer pipe to be rotatable in a direction around an axis of the insertion unit; an annular rotational operation member that is fixed to the proximal end side of the outer pipe and that rotates the outer pipe in the direction around the axis with respect to the operation unit body; a protection sheath that is inserted into the outer pipe and that is rotatable integrally with the outer pipe; a distal end optical system that is provided on a distal end side of the protection sheath; a flexible light guide that is disposed in a space between the outer pipe and the protection sheath and that includes a light emitting end on a distal end side of the outer pipe; an inner sheath that is inserted into the protection sheath and that is rotatable relative to the protection sheath in the direction around the axis; an image pickup unit that is provided on a distal end side of the inner sheath and that picks up an image of light passing through the distal end optical system; an external cable which is connected to a proximal end side of the operation unit body and into which the light guide is inserted from a distal end-side opening portion; and a fixing member that is disposed in the operation unit body, includes a fixing part for fixing a part of the light guide in a longitudinal direction, and is rotatable integrally with the rotational operation member in the direction around the axis. A light guide-insertion space is formed between the fixing part and the distal end-side opening portion in the operation unit body, and the light guide inserted into and disposed in the light guide-insertion space has a length longer than a linear distance between a proximal end of the fixing part and a center of the distal end-side opening portion.

According to the aspect of the present invention, it is preferable that the fixing member is connected to a proximal end side of the protection sheath and is rotatable integrally with the protection sheath around the axis.

According to the aspect of the present invention, it is preferable that the fixing member is formed of a ring-shaped member connected to the proximal end side of the protection sheath and includes the fixing part at a part of the ring-shaped member in a circumferential direction.

According to the aspect of the present invention, it is preferable that the endoscope further comprises a rotation stopper for regulating a rotation range of the fixing member around the axis.

According to the aspect of the present invention, it is preferable that the light guide has a length allowing a bent state to be maintained between the fixing part and the distal end-side opening portion in an axial direction of the insertion unit in the rotation range regulated by the rotation stopper.

According to the aspect of the present invention, it is preferable that the rotational operation member is provided on a distal end side of the operation unit body and is formed of an annular member rotatable in the direction around the axis with respect to the operation unit body.

According to the aspect of the present invention, it is preferable that the endoscope further comprises a signal cable connected to the image pickup unit and inserted into the inner sheath, and the signal cable is inserted into the external cable from the distal end-side opening portion.

According to the aspect of the present invention, it is preferable that the signal cable is a plurality of separated strands.

According to the aspect of the present invention, it is preferable that the endoscope further comprises: a pipe-like case that is connected to a proximal end side of the protection sheath in the operation unit body and that is disposed on a distal end side of the light guide-insertion space in an axial direction of the insertion unit; a partition wall that is provided in the case and that is perpendicular to an insertion axis of the insertion unit; and a magnet coupling which includes a first magnet provided on a distal end side in the axial direction and a second magnet provided on a proximal end side in the axial direction with the partition wall interposed therebetween and of which the first magnet is connected to a proximal end side of the inner sheath, and the magnet coupling and the case are rotatable relative to each other in the direction around the axis.

According to the aspect of the present invention, it is preferable that a distal end side of the external cable is connected to a proximal end side of the second magnet via a connecting member formed in a shape of a beam extending in the axial direction in the light guide-insertion space.

According to the aspect of the present invention, it is preferable that the endoscope further comprises a signal cable connected to the image pickup unit and is inserted into the inner sheath, and each of the first magnet and the second magnet is formed in a shape of a disk perpendicular to the insertion axis and includes an insertion hole into which the signal cable is to be inserted.

According to the aspect of the present invention, it is preferable that the fixing member is fixed to an outer peripheral surface of the case, and the fixing part of the fixing member is disposed on a distal end side of the insertion hole, which is formed in the second magnet, in the axial direction.

According to the aspect of the present invention, it is preferable that a peripheral edge portion of the distal end-side opening portion of the external cable is formed in a shape of a round surface.

According to the aspect of the present invention, it is preferable that the endoscope further comprises a proximal end optical system provided on the distal end side of the inner sheath and guiding light, which passes through the distal end optical system, to the image pickup unit, the image pickup unit includes an image pickup element that picks up an image of light, which is incident through the proximal end optical system, and that outputs an image pickup signal to a signal cable, and the distal end optical system is rotatable in the direction around the axis with respect to the proximal end optical system and the image pickup element.

According to the present invention, a structure is simple, and co-rotation of an external cable can be prevented.

### BRIEF DESCRIPTION OF THE DRAWINGS

FIG. 1 is a diagram showing a configuration of an endoscope system that comprises an oblique-viewing endoscope.

FIG. 2 is an enlarged cross-sectional view of a distal end portion of an insertion unit.

FIG. 3 is a cross-sectional view of an operation unit body.

FIG. 4 is a cross-sectional view of a protection sheath and a case.

FIG. 5 is an enlarged cross-sectional view of the case and a tubular portion.

FIG. 6 is a front view of a first magnet and a second magnet as viewed from a partition wall side.

FIG. 7 is a side view of the first magnet and the second magnet.

FIG. 8 is an internal structure diagram of an operation unit body showing a fixing member.

FIG. 9 is a diagram illustrating a posture of a light guide in a light guide-insertion space.

FIG. 10 is a diagram illustrating the posture of the light guide in the light guide-insertion space.

FIG. 11 is a diagram illustrating the posture of the light guide in the light guide-insertion space.

FIG. 12 is a diagram illustrating a length of the light guide in the light guide-insertion space.

FIG. 13 is a schematic diagram showing a configuration of a rotation stopper.

### DESCRIPTION OF THE PREFERRED EMBODIMENTS

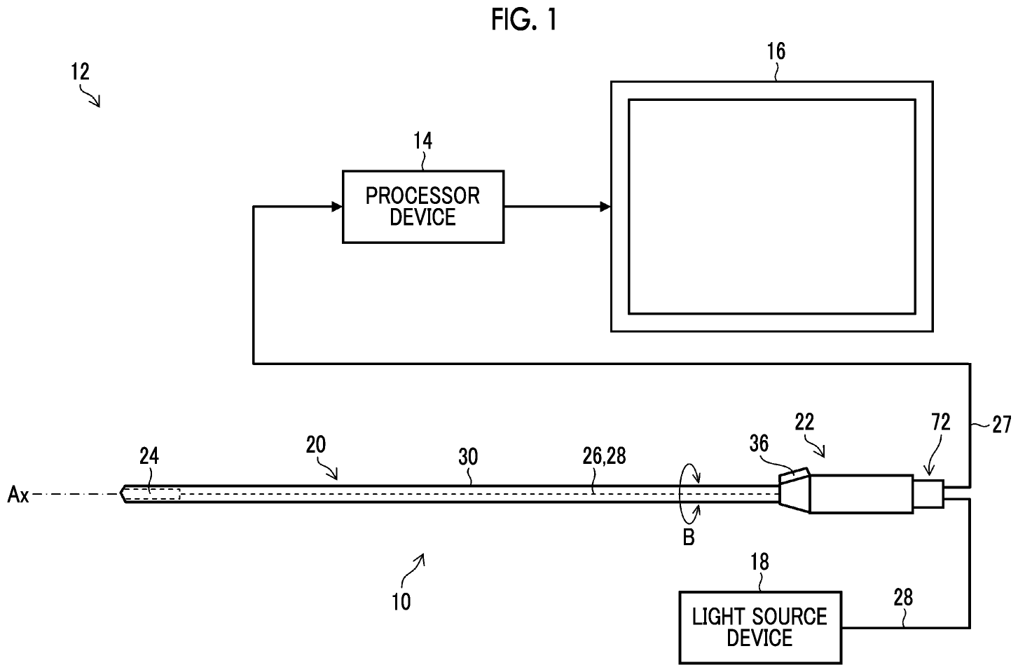

FIG. 1 is a diagram showing the configuration of an endoscope system 12 that comprises an oblique-viewing endoscope 10. As shown in FIG. 1, the endoscope system 12 comprises the oblique-viewing endoscope 10, a processor device 14, a monitor 16, and a light source device 18. The oblique-viewing endoscope 10 is an example of an endoscope of the present invention.

The oblique-viewing endoscope 10 shown in FIG. 1 is a so-called rigid endoscope, and comprises an insertion unit 20 and an operation unit body 22. The insertion unit 20 is an example of an insertion unit of the present invention. The operation unit body 22 is a part to be gripped by a practitioner (not shown) during the operation of the oblique-viewing endoscope 10, and is formed in a tubular shape. The operation unit body 22 is an example of an operation unit body of the present invention.

The insertion unit 20 is formed substantially in the shape of a pipe (in a substantially tubular shape), and is inserted into a patient's body. The insertion unit 20 has a distal end, a proximal end, and an insertion axis Ax.

The insertion unit 20 comprises an outer pipe 30 forming the insertion unit 20. The operation unit body 22 supports the outer pipe 30 to allow the outer pipe 30 to be rotatable in a direction around the insertion axis Ax (a circumferential direction of the insertion unit 20 indicated by an arrow B, hereinafter, simply referred to as a “direction B around the axis”). The outer pipe 30 is an example of an outer pipe of the present invention.

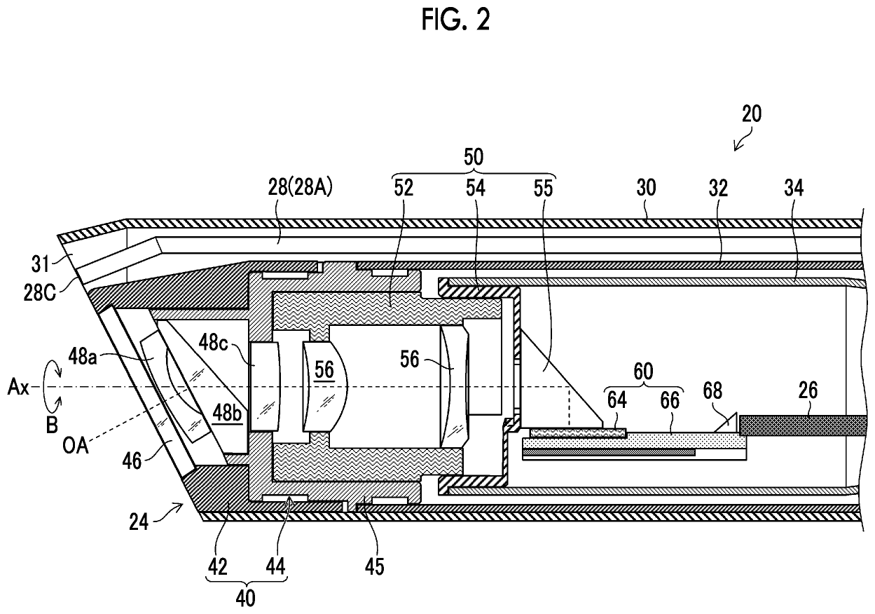

An annular knob 36 is fixed to a proximal end side of the outer pipe 30. The knob 36 is a member that is used to rotate the outer pipe 30 in the direction B around the axis with respect to the operation unit body 22. In a case where the outer pipe 30 is operated to rotate using the knob 36, a visual field direction (observation direction, see an optical axis OA shown in FIG. 2) of the oblique-viewing endoscope 10 can be rotated in the direction B around the axis. The knob 36 is an example of a rotational operation member of the present invention.

A camera unit 24 to be described later is provided in a distal end portion of the insertion unit 20. Further, a first signal cable 26 and a light guide 28 are inserted into the insertion unit 20.

The first signal cable 26 connects the camera unit 24 to the processor device 14 together with a second signal cable 27 to be described later. That is, a distal end side of the first signal cable 26 is connected to the camera unit 24, and a proximal end side of the first signal cable 26 is connected to a distal end side of the second signal cable 27 in the operation unit body 22. A proximal end side of the second signal cable 27 is connected to the processor device 14. The first signal cable 26 and the second signal cable 27 are an example of a signal cable of the present invention. In this embodiment, a multicore cable in which a plurality of strands (signal lines) are bundled, a shield conductor is provided around the strands, and the strands and the shield conductor are housed in a tubular sheath is exemplified as each of the first signal cable 26 and the second signal cable 27.

The light guide 28 has a light emitting end 28C (see FIG. 2) on the distal end side thereof, and the light emitting end 28C is disposed on the distal end side of the outer pipe 30. Further, the light guide 28 has a light incident end (not shown) on the proximal end side thereof, and the light incident end is connected to the light source device 18. For example, one optical cable in which a plurality of optical fibers are bundled is employed as the light guide 28, and has flexibility. The light guide 28 is an example of a light guide of the present invention.

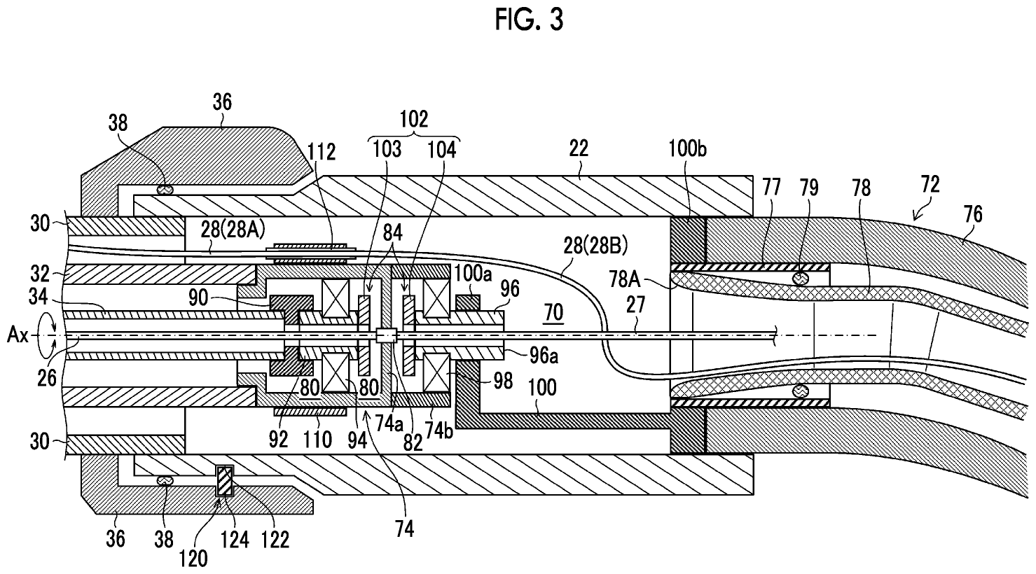

As described in detail later, the operation unit body 22 includes an airtight space and a non-airtight space therein, and the proximal end side of the first signal cable 26 and the distal end side of the second signal cable 27 are connected to each other at a boundary between both the spaces (see FIG. 3). Accordingly, the camera unit 24 and the processor device 14 are electrically connected to each other via the first signal cable 26 and the second signal cable 27.

The processor device 14 generates an observation image (video) of the inside of the patient's body on the basis of image pickup signals, which are input from the camera unit 24 through the first signal cable 26 and the second signal cable 27, and causes the monitor 16 to display this observation image.

The light source device 18 supplies illumination light to the light guide 28. Accordingly, illumination light is emitted from the light emitting end 28C (see FIG. 2) of the light guide 28 that is provided on the distal end side of the outer pipe 30.

FIG. 2 is an enlarged cross-sectional view of the distal end portion of the insertion unit 20. As shown in FIG. 2, the insertion unit 20 comprises the outer pipe 30, a protection sheath 32, and an inner sheath 34 that are formed substantially in the shape of a pipe parallel to the insertion axis Ax. The outer pipe 30 forms an outer peripheral wall of the insertion unit 20. A distal end side-opening of the outer pipe 30 is inclined from a posture perpendicular to the insertion axis Ax.

The protection sheath 32 is inserted into and disposed in the outer pipe 30. A distal end optical system 40 of the camera unit 24 is provided on a distal end side of the protection sheath 32. Further, as described in detail later, a proximal end side of the protection sheath 32 is connected to a pipe-like case 74 (see FIG. 3) in the operation unit body 22. Furthermore, a space 31 in which the light guide 28 is to be disposed is formed between the inner peripheral surface of the outer pipe 30 and the outer peripheral surface of the protection sheath 32. The light guide 28 is inserted into the space 31 and is fixed to the inner peripheral surface of the outer pipe 30 and to the outer peripheral surface of the protection sheath 32. The protection sheath 32 is an example of a protection sheath of the present invention.

The inner sheath 34 is inserted into and disposed in the protection sheath 32. The first signal cable 26 is inserted into the inner sheath 34. A proximal end optical system 50 and an image pickup unit 60 of the camera unit 24 are provided on a distal end side of the inner sheath 34. Further, as described in detail later, a proximal end side of the inner sheath 34 is connected to a connection member 90 (see FIG. 3) in the operation unit body 22. The inner sheath 34 is an example of an inner sheath of the present invention.

As shown in FIG. 2, the camera unit 24 comprises the distal end optical system 40, the proximal end optical system 50, and the image pickup unit 60. Reference character OA shown in FIG. 2 denotes the optical axis of the optical system of the camera unit 24.

The distal end optical system 40 is provided on the distal end side of the protection sheath 32. The distal end optical system 40 is an oblique-viewing optical system that refracts light, which is incident in a direction inclined with respect to the insertion axis Ax, in a direction parallel to the insertion axis Ax and that guides the light to the proximal end optical system 50. The distal end optical system 40 includes a distal end portion body 42 and a distal end lens barrel 44 that is provided in the distal end portion body 42. The distal end optical system 40 is an example of a distal end optical system of the present invention.

The distal end portion body 42 forms the distal end portion of the insertion unit 20 (protection sheath 32) and is a cap that covers the distal end lens barrel 44. Further, the distal end portion body 42 is formed substantially in the shape of a pipe parallel to the insertion axis Ax. Furthermore, a cover glass 46, which is in an inclined posture corresponding to an inclination angle of an objective lens 48a provided in the distal end lens barrel 44, is provided at a distal end side-opening portion of the distal end portion body 42.

Further, the distal end portion body 42 is fixed to the inner peripheral surface of the outer pipe 30. Accordingly, in a case where the outer pipe 30 is rotated in the direction B around the axis, the distal end optical system 40 and the protection sheath 32 are integrally rotated in the direction B around the axis together with the outer pipe 30.

The objective lens 48a, a prism 48b, and a lens 48c are housed in the distal end lens barrel 44. The objective lens 48a is inclined from a posture perpendicular to the insertion axis Ax and faces the cover glass 46. The objective lens 48a emits light, which is incident through the cover glass 46, toward the prism 48b. The prism 48b refracts light incident from the objective lens 48a, that is, light incident in a direction inclined with respect to the insertion axis Ax, in a direction parallel to the insertion axis Ax and then emits the light toward the lens 48c. The lens 48c is in a posture perpendicular to the insertion axis Ax, and emits light incident from the prism 48b toward lenses 56 that are provided in a proximal end lens barrel 52 of the proximal end optical system 50.

The configuration of an optical system provided in the distal end lens barrel 44 is not particularly limited as long as light incident in a direction inclined with respect to the insertion axis Ax can be guided into the proximal end lens barrel 52.

A tubular portion 45, which extends toward the proximal end side of the distal end lens barrel 44, is formed at the distal end lens barrel 44. The tubular portion 45 is externally fitted to be rotatable relative to a distal end portion of the proximal end lens barrel 52 in the direction B around the axis. Accordingly, the proximal end lens barrel 52 is fitted to be rotatable relative to the distal end lens barrel 44 in the direction B around the axis.

The proximal end optical system 50 is provided on the distal end side of the inner sheath 34, and guides light, which is incident from the distal end lens barrel 44, to the image pickup unit 60. The proximal end optical system 50 includes the proximal end lens barrel 52, a holder 54, and a prism 55. The proximal end optical system 50 is an example of a proximal end optical system of the present invention.

A proximal end side of the proximal end lens barrel 52 is fixed to the distal end side of the inner sheath 34 via the holder 54. Further, the distal end side of the proximal end lens barrel 52 is fitted to be rotatable relative to a proximal end-side opening portion of the tubular portion 45 in the direction B around the axis as described above. Accordingly, one of the distal end lens barrel 44 and the proximal end lens barrel 52 is rotatable relative to the other thereof in the direction B around the axis. As a result, the inner sheath 34 inserted into the protection sheath 32 is rotatable relative to the protection sheath 32 in the direction B around the axis.

A plurality of lenses 56 having an optical axis OA parallel to the insertion axis Ax are provided in the proximal end lens barrel 52. Each lens 56 emits light, which is incident from the distal end lens barrel 44, toward the prism 55.

The holder 54 is formed substantially in the shape of a pipe parallel to the insertion axis Ax, and is fixed to the distal end side of the inner sheath 34. Further, the holder 54 is externally fitted and fixed to the proximal end side of the proximal end lens barrel 52. Accordingly, since the inner sheath 34 and the proximal end lens barrel 52 are connected to each other by the holder 54, the inner sheath 34, the holder 54, and the proximal end lens barrel 52 are integrally rotatable relative to the protection sheath 32 in the direction B around the axis.

The prism 55 is held at a proximal end-side opening portion of the holder 54, and the image pickup unit 60 is held via the prism 55. For this reason, the image pickup unit 60 is rotatable relative to the protection sheath 32 in the direction B around the axis integrally with the inner sheath 34 and the proximal end lens barrel 52 via the holder 54 and the prism 55.

The prism 55 refracts light, which is incident through the proximal end lens barrel 52, by an angle of 90°. A mirror may be used instead of the prism 55.

The image pickup unit 60 picks up the image of the light (observation image) that passes through the distal end optical system 40 and the proximal end optical system 50 and that is reflected by the prism 55. The image pickup unit 60 comprises an image pickup element 64 and a circuit board 66. The image pickup unit 60 is an example of an image pickup unit of the present invention.

The image pickup element 64 is fixed to the prism 55 in a state where the image pickup element 64 is mounted on the circuit board 66, and is mounted on the holder 54 via the prism 55. Further, the image pickup element 64 picks up the image of the light, which is refracted by the prism 55, and outputs image pickup signals. A charge coupled device (CCD) image sensor or a complementary metal oxide semiconductor (CMOS) image sensor is used as the image pickup element 64. The image pickup element 64 is an example of an image pickup element of the present invention.

The circuit board 66 controls the drive of the image pickup element 64. Further, the distal end side of the first signal cable 26 is connected to the circuit board 66 via a connector 68. Furthermore, the circuit board 66 outputs the image pickup signals of the image pickup element 64 to the first signal cable 26 via the connector 68.

FIG. 3 is a cross-sectional view of the operation unit body 22. As shown in FIG. 3, the operation unit body 22 is formed in the shape of a pipe parallel to the insertion axis Ax.

The annular knob 36 fixed to the proximal end side of the outer pipe 30 is provided on the distal end side of the operation unit body 22. For example, the knob 36 is rotatably provided on the outer peripheral surface of a distal end portion of the operation unit body 22 through a seal ring 38. Accordingly, the knob 36 is formed of an annular member that is rotatable relative to the operation unit body 22 in the direction B around the axis. In a case where the knob 36 is operated to rotate in the direction B around the axis, the outer pipe 30 is rotated relative to the operation unit body 22 in the direction B around the axis, and the protection sheath 32 and the distal end optical system 40 (the distal end portion body 42 and the distal end lens barrel 44, see FIG. 2) are rotated in the same direction via the outer pipe 30. Accordingly, the visual field direction (observation direction) of the oblique-viewing endoscope 10 can be changed.

The proximal end sides of the protection sheath 32 and the inner sheath 34 are inserted into the operation unit body 22 from a distal end side-opening portion of the operation unit body 22. Further, an external cable 72 to be described later is connected to a proximal end side of the operation unit body 22. Furthermore, a light guide-insertion space 70 to be described later is formed in the operation unit body 22. Moreover, the case 74 is provided in the operation unit body 22. The case 74 is disposed on the distal end side of the light guide-insertion space 70.

A distal end side of the external cable 72 is connected to the proximal end side of the operation unit body 22, and is provided integrally with the operation unit body 22. The external cable 72 includes a cable body 76 that forms a sheath, and a connection tube 78 that is inserted into and disposed in the cable body 76. The external cable 72 is an example of an external cable of the present invention.

A distal end side of the connection tube 78 is formed in a funnel shape, and the second signal cable 27 and the light guide 28 are inserted into the connection tube 78 from a distal end-side opening portion 78A of which the diameter is increased. Since a peripheral edge portion of the distal end-side opening portion 78A is formed in the shape of a round surface, damage to the second signal cable 27 and to the light guide 28 is prevented in a case where the second signal cable 27 and the light guide 28 are in contact with the distal end-side opening portion 78A. The distal end-side opening portion 78A is an example of a distal end-side opening portion of the present invention.

A pipe-like metal fitting 77 is fixed to the inner peripheral surface of a distal end portion of the cable body 76, and the connection tube 78 is fixed to the inner peripheral surface of the metal fitting 77 via an O-ring 79. As described in detail later, the connection tube 78 is connected to a magnet coupling 102 via a connecting beam 100 and a bearing receiving member 96. The configuration of the external cable 72 has been described above, but this configuration is an example. For example, a configuration in which the connection tube 78 is not provided and a distal end-side opening portion 78A is formed on the distal end side of the cable body 76 can also be applied as another configuration of the external cable 72.

Next, a configuration that allows the first signal cable 26 and the second signal cable 27 to be inserted into and disposed in the operation unit body 22 will be described.

As shown in FIG. 3, the case 74 is formed substantially in the shape of a pipe parallel to the insertion axis Ax to have a diameter smaller than an inner diameter of the operation unit body 22 and is housed in the operation unit body 22. The case 74 is supported in the internal space of the operation unit body 22 by the protection sheath 32, the external cable 72, and the like. A distal end side of the case 74 is connected to the proximal end portion of the protection sheath 32. Accordingly, in a case where the outer pipe 30 is rotated relative to the operation unit body 22 in the direction B around the axis, this rotational force is transmitted to the distal end optical system 40, the protection sheath 32, and the case 74. As a result, the case 74 is rotated in the same direction as the outer pipe 30. The case 74 is an example of a case of the present invention.

The proximal end side of the inner sheath 34 and the proximal end side of the first signal cable 26 are disposed in the case 74. Further, a partition wall 74a perpendicular to the insertion axis Ax is provided in the case 74, for example, in the proximal end-side opening portion of the case 74. The partition wall 74a closes the proximal end-side opening portion of the case 74. The partition wall 74a is an example of a partition wall of the present invention.

Furthermore, a tubular portion 74b parallel to the insertion axis Ax is provided on the proximal end side of the case 74. The tubular portion 74b is formed to have the same diameter as the case 74, but may be formed to have a diameter different from the diameter of the case 74. Further, the tubular portion 74b may be formed integrally with the case 74. In this case, the proximal end side of the case 74 functions as a tubular portion 74b. The distal end side of the second signal cable 27 is disposed in the case 74 and the tubular portion 74b in addition to a part of a connecting unit 84 to be described later.

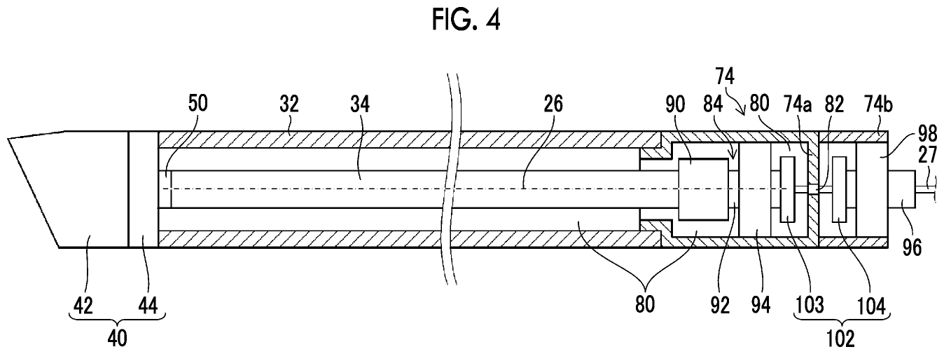

FIG. 4 is a cross-sectional view of the protection sheath 32 and the case 74. A sealed space 80(airtight space) is formed in the protection sheath 32 and the case 74 as shown in FIG. 4, and the inner sheath 34, the image pickup unit 60 (see FIG. 2), the first signal cable 26, and the like are disposed in the sealed space 80. A distal end side of the sealed space 80 is defined by the distal end optical system 40. Further, a proximal end side of the sealed space 80 is defined by the partition wall 74a. Accordingly, the moisture-proof property of the camera unit 24 is improved, so that fogging is prevented.

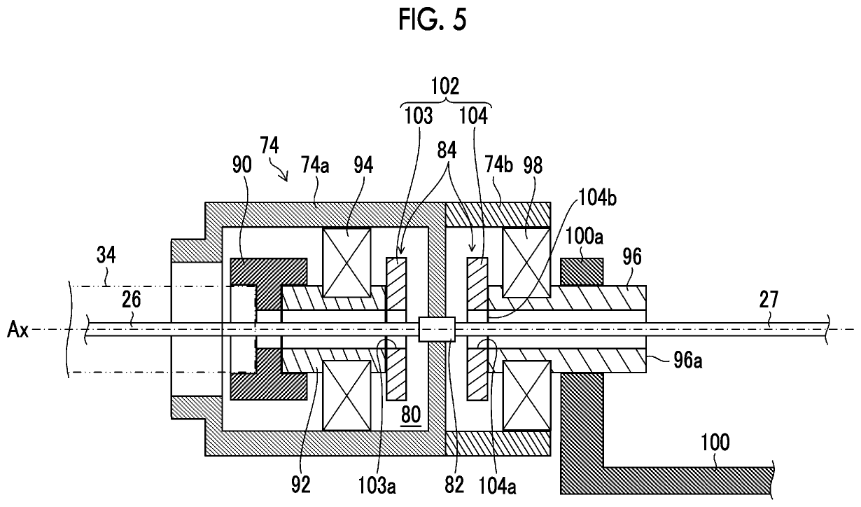

FIG. 5 is an enlarged cross-sectional view of the case 74 and the tubular portion 74b. As shown in FIGS. 3 to 5, the partition wall 74a already described, an air-tight connector 82, and a connecting unit 84 are provided in the case 74 and the tubular portion 74b.

The air-tight connector 82 is provided to pass through the inside and the outside of the sealed space 80 and to be rotatable relative to the partition wall 74a in the direction B around the axis. The air-tight connector 82 electrically connects the proximal end side of the first signal cable 26 provided in the case 74 (in the sealed space 80) to the distal end side of the second signal cable 27 provided in the tubular portion 74b (outside the sealed space 80). Accordingly, the first signal cable 26 and the second signal cable 27 are inserted into and disposed in the operation unit body 22. In a case where the first signal cable 26 and the second signal cable 27 are torsionally deformable in the direction B around the axis, for example, in a case where each of the first signal cable 26 and the second signal cable 27 is formed of a plurality of separated strands, the air-tight connector 82 may be fixed to the partition wall 74a.

The connecting unit 84 is provided in the case 74 and the tubular portion 74b to be rotatable relative to the case 74 and the tubular portion 74b in the direction B around the axis. The first signal cable 26 and the second signal cable 27 are inserted into the connecting unit 84. The connecting unit 84 magnetically connects the proximal end side of the inner sheath 34 provided in the case 74 (in the sealed space 80) to the distal end side of the external cable 72 (see FIG. 3) provided outside the sealed space 80 with the partition wall 74a interposed therebetween.

The connecting unit 84 comprises a connection member 90, a bearing receiving member 92, and a bearing 94. Further, the connecting unit 84 comprises a bearing receiving member 96, a bearing 98, a connecting beam 100, and a magnet coupling 102 in addition to the above-mentioned members.

The connection member 90 and the bearing receiving member 92 are provided in the case 74 (in the sealed space 80), and are formed substantially in the shape of a pipe parallel to the insertion axis Ax. Further, the first signal cable 26 is inserted into the connection member 90 and the bearing receiving member 92.

The connection member 90 connects the proximal end side of the inner sheath 34 to a distal end side of the bearing receiving member 92 in the case 74 (in the sealed space 80). Accordingly, the distal end side of the bearing receiving member 92 is connected to the proximal end side of the inner sheath 34 via the connection member 90.

The distal end side of the bearing receiving member 92 is connected to the connection member 90 as described above, and a proximal end side thereof is fixed to a first magnet 103 of the magnet coupling 102. Further, the bearing 94, which is to be inscribed in the case 74, is fixed to the outer peripheral surface of the bearing receiving member 92. Accordingly, the bearing receiving member 92 and the first magnet 103 are held in the case 74 to be rotatable relative to the case 74 in the direction B around the axis. Publicly known various radial bearings, such as a ball bearing and a roller bearing, are used as the bearing 94.

Furthermore, a rolling body (a ball or a roller), which is a component of the bearing 94, is made of a non-magnetic material. Since the rolling body of the bearing 94 is made of a non-magnetic material, it is possible to prevent the magnetic force of the first magnet 103 from acting on the rolling body. As a result, the bearing receiving member 92 and the first magnet 103 can be smoothly rotated relative to the case 74. Examples of the non-magnetic material can include ceramics, non-magnetic metal (for example, stainless steel), and a resin. Not only the rolling body but also other components (an inner race, an outer race, and a retainer) of the bearing 94 may be made of a non-magnetic material.

The bearing receiving member 96 is provided in the tubular portion 74b (outside the sealed space 80). The bearing receiving member 96 is formed substantially in the shape of a pipe parallel to the insertion axis Ax, and the second signal cable 27 is inserted into the bearing receiving member 96.

A distal end portion of the bearing receiving member 96 is fixed to a second magnet 104 of the magnet coupling 102 in the tubular portion 74b, and a proximal end portion thereof is connected to the connecting beam 100. Further, a bearing 98, which is to be inscribed in the tubular portion 74b, is fixed to the outer peripheral surface of the bearing receiving member 96. Accordingly, the bearing receiving member 96 and the second magnet 104 are held in the tubular portion 74b to be rotatable relative to the tubular portion 74b in the direction B around the axis. Publicly known various radial bearings are also used as the bearing 98 as in the case of the bearing 94.

Furthermore, a rolling body (a ball or a roller), which is a component of the bearing 98, is made of a non-magnetic material as in the case of the bearing 94. Since the rolling body of the bearing 98 is made of a non-magnetic material, it is possible to prevent the magnetic force of the second magnet 104 from acting on the rolling body. As a result, the bearing receiving member 96 and the second magnet 104 can be smoothly rotated relative to the tubular portion 74b. Examples of the non-magnetic material can include ceramics, non-magnetic metal (for example, stainless steel), and a resin. Not only the rolling body but also other components (an inner race, an outer race, and a retainer) of the bearing 98 may be made of a non-magnetic material.

According to the above-mentioned configuration, the endoscope 10 according to an embodiment comprises the protection sheath 32 that forms the insertion unit 20, the pipe-like case 74 that is connected to the proximal end side of the protection sheath 32, the distal end optical system 40 that is provided at the distal end of the protection sheath 32 and that defines the distal end side of the sealed space 80 formed in the protection sheath 32 and the case 74, the partition wall 74a that is provided in the case 74, is perpendicular to the insertion axis Ax of the insertion unit 20, and defines the proximal end side of the sealed space 80, the inner sheath 34 that is inserted into the protection sheath 32 and that is rotatable relative to the protection sheath 32 in the direction B around the insertion axis Ax, the image pickup unit 60 that is provided at the distal end of the inner sheath 34 and that picks up the image of light passing through the distal end optical system 40, and the magnet coupling 102 which includes the first magnet 103 provided in the sealed space 80 and the second magnet 104 provided outside the sealed space 80 with the partition wall 74a interposed therebetween and of which the first magnet 103 is connected to the proximal end side of the inner sheath 34. The endoscope 10 has a configuration in which the magnet coupling 102 and the case 74 are rotatable relative to each other in the direction B around the axis. Further, each of the rolling body of the bearing 94 that supports the first magnet 103 to allow the first magnet 103 to be rotatable relative to the case 74 and the rolling body of the bearing 98 that supports the second magnet 104 to allow the second magnet 104 to be rotatable relative to the tubular portion 74b is made of a non-magnetic material.

As shown in FIG. 3, the connecting beam 100 is formed in the shape of a beam that extends in the direction of the insertion axis Ax in the light guide-insertion space 70 to be described later. The connecting beam 100 includes a ring portion 100a provided on a distal end side thereof and a ring portion 100b provided on a proximal end side thereof, and the ring portion 100a is externally fitted to a proximal end side of the bearing receiving member 96, and the ring portion 100b is externally fitted to a distal end side of the metal fitting 77. As a result, a proximal end side of the second magnet 104 via the bearing receiving member 96 and the distal end side of the external cable 72 via the metal fitting 77 are connected to each other via the connecting beam 100. In other words, the distal end side of the external cable 72 is connected to the proximal end side of the second magnet 104 via the connecting beam 100. The connecting beam 100 is an example of a connecting member of the present invention.

The magnet coupling 102 includes the first magnet 103 provided in the case 74 (in the sealed space 80) and the second magnet 104 provided in the tubular portion 74b (outside the sealed space 80) with the partition wall 74a interposed therebetween. The magnet coupling 102 is a magnetic connecting member that magnetically connects the bearing receiving member 92 (inner sheath 34) to the bearing receiving member 96 (external cable 72), and is an example of a magnet coupling of the present invention. Further, the first magnet 103 is an example of a first magnet of the present invention, and the second magnet 104 is an example of a second magnet of the present invention.

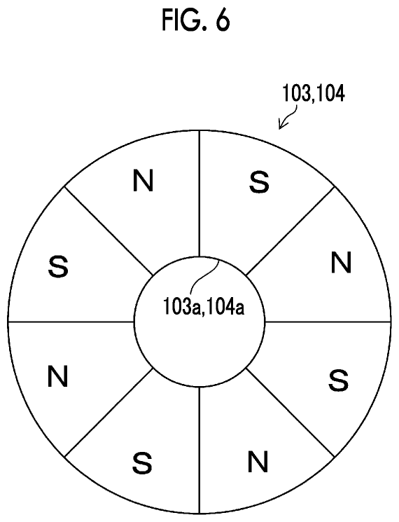

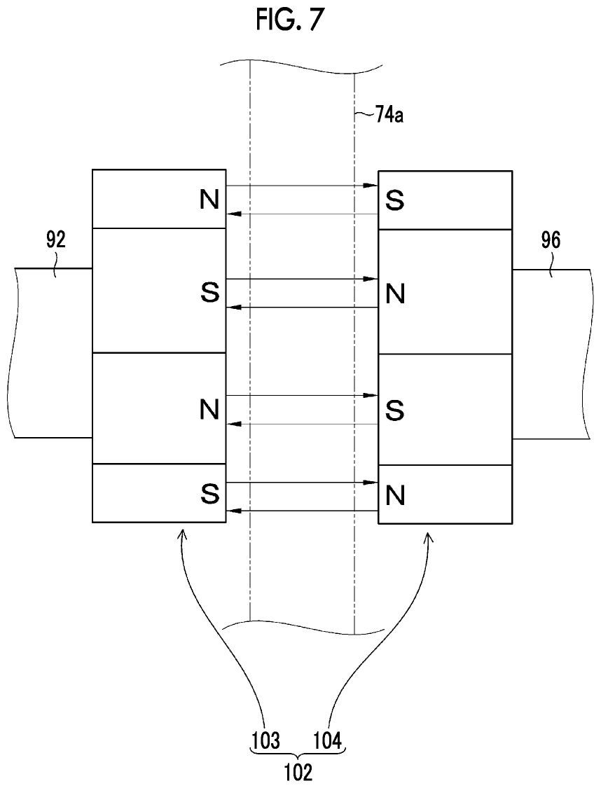

FIG. 6 is a front view of the first magnet 103 and the second magnet 104 as viewed from a partition wall 74a side. FIG. 7 is a side view of the first magnet 103 and the second magnet 104. As shown in FIG. 6, the first magnet 103 and the second magnet 104 have the shape of a disk (the shape of a ring) parallel to the partition wall 74a (perpendicular to the insertion axis Ax). An insertion hole 103a into which the first signal cable 26 is to be inserted is formed at the central portion of the first magnet 103, and an insertion hole 104a into which the second signal cable 27 is to be inserted is formed at the central portion of the second magnet 104. Further, each of the first magnet 103 and the second magnet 104 is a so-called single-sided multipole magnet, and a plurality of sets of N poles and S poles are formed on a surface side thereof facing the partition wall 74a at regular angular intervals in the direction around the axis.

Each of the first magnet 103 and the second magnet 104 is not limited to a single-sided multipole magnet and may be a double-sided multipole magnet, and the number of poles is not particularly limited as long as two or more poles are provided. Furthermore, the shape of each of the first magnet 103 and the second magnet 104 is not limited to the shape of a disk, and the shape of any member parallel to the partition wall 74a, such as a polygonal shape, may be employed.

As shown in FIG. 7, the first magnet 103 and the second magnet 104 are disposed with the partition wall 74a interposed therebetween so that individual N poles of any one of the first magnet 103 or the second magnet 104 face individual S poles of the other thereof and individual S poles of one thereof face individual N poles of the other thereof. Accordingly, the first magnet 103 and the second magnet 104 are magnetically connected to each other in a thrust direction of the insertion axis Ax (a direction parallel to the insertion axis Ax) with the partition wall 74a interposed therebetween. As a result, the inner sheath 34 and the external cable 72 are magnetically connected to each other via the magnet coupling 102.

Since the inner sheath 34 and the external cable 72 are magnetically connected to each other via the magnet coupling 102, torque (stop torque, rotational torque) can be transmitted to the inner sheath 34 from the external cable 72. Accordingly, in a case where a practitioner rotationally operates the outer pipe 30 using the knob 36, the rotation (co-rotation) of the protection sheath 32 and the inner sheath 34 (the proximal end optical system 50 and the image pickup unit 60) in the direction B around the axis is prevented, that is, the posture of the inner sheath 34 in the direction B around the axis is maintained by the magnet coupling 102.

Next, a configuration that allows the light guide 28 to be inserted into and disposed in the operation unit body 22 will be described.

As shown in FIG. 3, a fixing member 110 is disposed in the operation unit body 22. The fixing member 110 includes a fixing part 112 for fixing a part of the light guide 28 in a longitudinal direction.

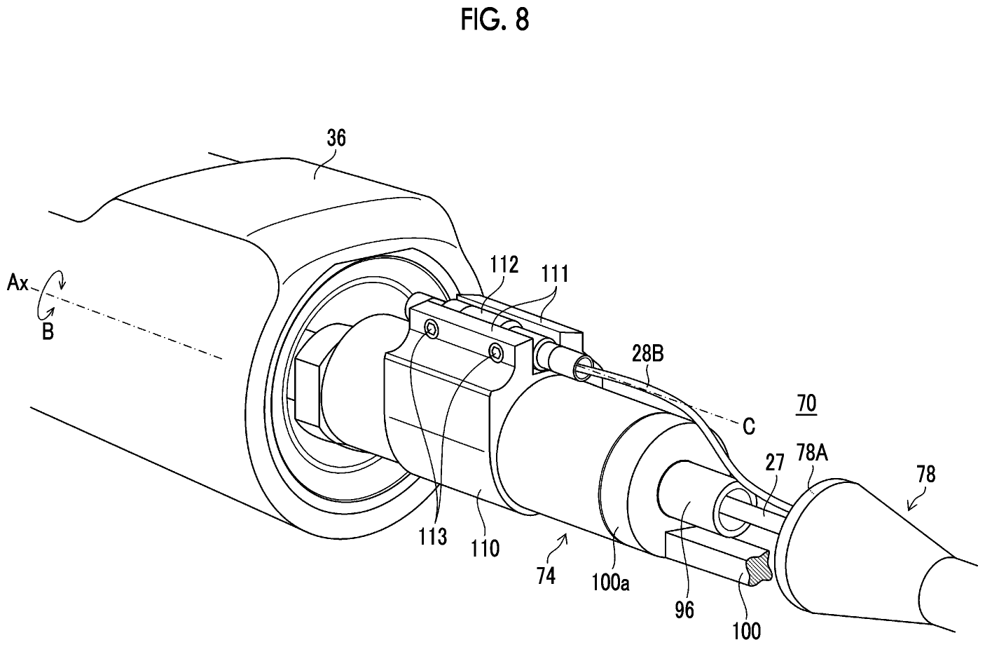

FIG. 8 is an internal structure diagram of the operation unit body 22 showing the configuration of the fixing member 110. As shown in FIG. 8, the fixing member 110 is formed as a ring-shaped member and is connected to the outer peripheral surface of the case 74 connected to the proximal end side of the protection sheath 32 (see FIG. 3). Accordingly, the fixing member 110 is connected to the protection sheath 32 via the case 74 and is rotated integrally with the protection sheath 32 and the case 74 in the direction B around the axis. The fixing member 110 is an example of a fixing member of the present invention.

The fixing member 110 includes the fixing part 112 at a part thereof in the circumferential direction. The fixing part 112 is formed as a pipe-like member into which the light guide 28 can be inserted, and is fixed in a gap between a pair of flanges 111 and 111, which protrudes from the outer peripheral surface of the fixing member 110, by screws 113. Accordingly, the fixing part 112 is disposed so that a central axis C of the fixing part 112 is substantially parallel to the insertion axis Ax. The light guide 28 is inserted into the fixing part 112, so that a part of the light guide 28 in the longitudinal direction is fixed by the fixing part 112. The fixing part 112 is an example of a fixing part of the present invention.

The ring-shaped member has been described as the fixing member 110 by way of example in this embodiment, but the shape of the fixing member 110 is not particularly limited as long as the fixing member 100 is rotatable integrally with the protection sheath 32 in the direction B around the axis. Likewise, the pipe-like member has been described as the fixing part 112 by way of example, but the shape of the fixing part 112 is not particularly limited as long as the fixing part 112 can fix a part of the light guide 28 in the longitudinal direction. Further, the fixing member 110 and the fixing part 112 have been described as separate bodies by way of example in this embodiment, but the fixing member 110 and the fixing part 112 may be integrated with each other. Furthermore, an aspect in which the fixing member 110 is connected to the proximal end side of the protection sheath 32 via the case 74 has been described by way of example in this embodiment, but the fixing member 110 may be directly connected to the proximal end side of the protection sheath 32.

In a case where a part of the light guide 28 is fixed by the fixing part 112 in the operation unit body 22 as in this embodiment, the light guide 28 is divided into a light guide 28 (hereinafter, referred to as a “light guide 28A”) that is disposed toward the distal end side from the fixing part 112 (insertion unit 20 side) and a light guide 28 (hereinafter, referred to as “light guide 28B”) that is disposed toward the proximal end side from the fixing part 112 (external cable 72 side). A configuration in which one light guide 28 is divided into the light guide 28A and the light guide 28B with the fixing part 112 interposed therebetween has been described by way of example in this embodiment, but a configuration in which two light guides 28A and 28B are used and a proximal end side of the light guide 28A and a distal end side of the light guide 28B are connected to each other via the fixing part 112 can also be applied.

Here, since the light guide 28A is fixed to the inner peripheral surface of the outer pipe 30 and to the outer peripheral surface of the protection sheath 32, the outer pipe 30 and the protection sheath 32 are integrally rotated together with the fixing member 110 (fixing part 112) in a case where the outer pipe 30 and the protection sheath 32 are rotated. Accordingly, the light guide 28A is not rubbed against the inner peripheral surface of the outer pipe 30 and the outer peripheral surface of the protection sheath 32 during rotation, so that damage caused by rubbing is prevented.

Meanwhile, the light guide 28B is inserted into and disposed in the light guide-insertion space 70 formed between the fixing part 112 and the distal end-side opening portion 78A in the operation unit body 22 (see FIG. 3). Further, in a case where the fixing member 110 is rotated in the direction B around the axis by the knob 36, the light guide 28B is inserted into and disposed in the light guide-insertion space 70 in a state where tension is not applied to the light guide 28B. An aspect in which the light guide 28B is inserted into and disposed in the light guide-insertion space 70 will be specifically described below.

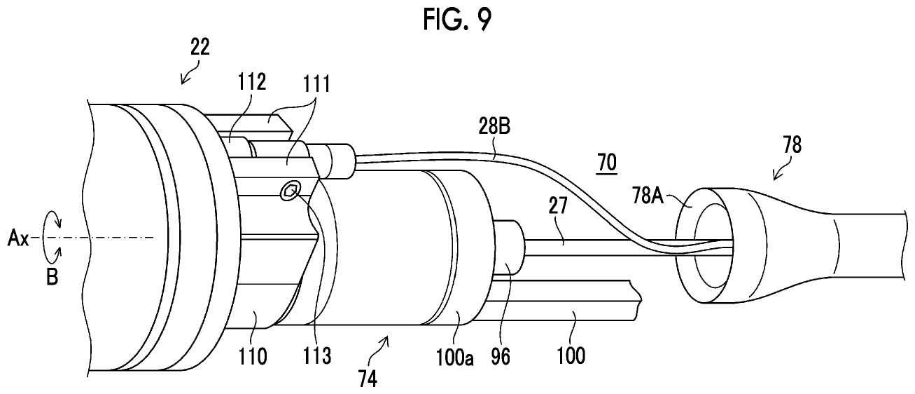

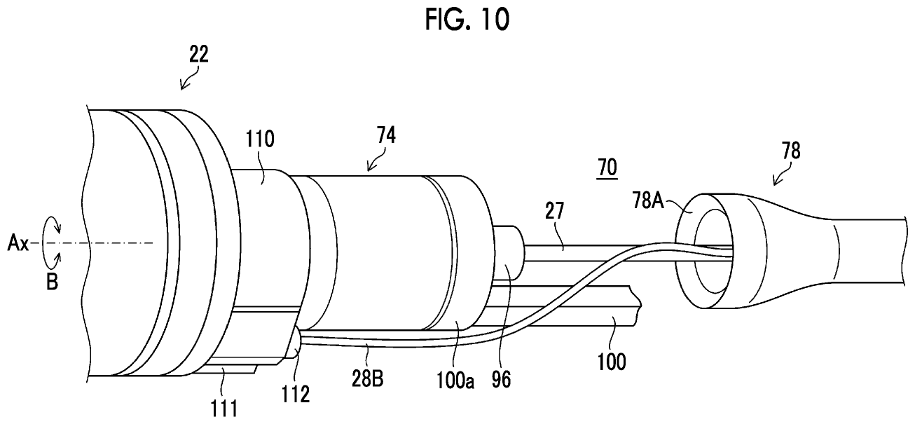

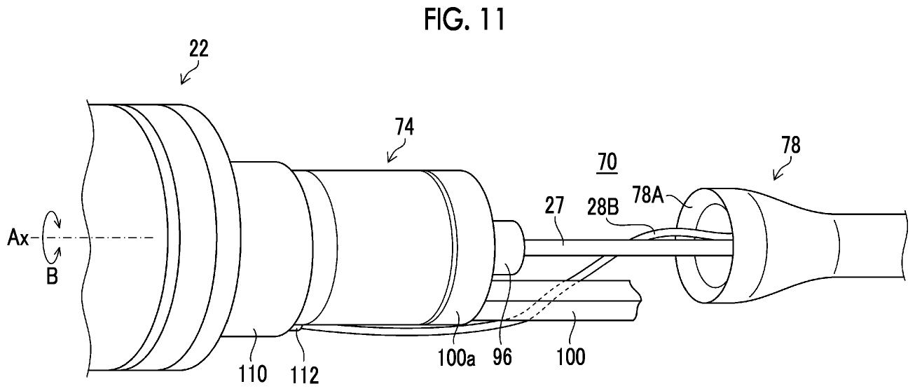

FIGS. 9, 10, and 11 are diagrams illustrating aspects in which the light guide 28B is inserted into and disposed in the light guide-insertion space 70 (hereinafter, referred to as the “postures” of the light guide 28B), respectively. That is, FIG. 9 shows the posture of the light guide 28B in a case where the fixing part 112 is positioned at a middle position in a rotation range to be described later of the fixing member 110. FIG. 10 shows the posture of the light guide 28B in a case where the fixing member 110 (fixing part 112) is rotated leftward (in a counterclockwise direction) as the fixing member 110 is viewed from the proximal end side of the operation unit body 22. FIG. 11 shows the posture of the light guide 28B in a case where the fixing member 110 (fixing part 112) is rotated rightward (in a clockwise direction) as the fixing member 110 is viewed from the proximal end side of the operation unit body 22.

As shown in FIGS. 9 to 11, the posture of the light guide 28B in the light guide-insertion space 70 varies depending on the rotational position of the fixing part 112. However, regardless of the posture of the light guide 28B, the light guide 28B is maintained in the light guide-insertion space 70 in a state where tension is not applied.

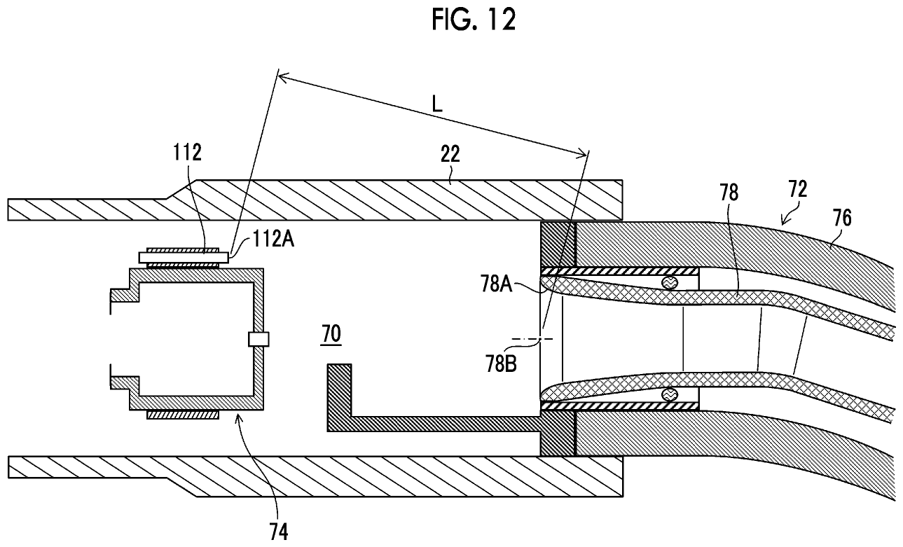

In order to maintain a bent state in this way, the light guide 28B has a length that allows a bent state to be maintained between the fixing part 112 and the distal end-side opening portion 78A in the direction of the insertion axis Ax in the rotation range of the fixing member 110. In other words, as shown in a diagram showing the length of the light guide 28B in the light guide-insertion space 70 shown in FIG. 12, the light guide 28B inserted into and disposed in the light guide-insertion space 70 has a length longer than a linear distance L between a proximal end 112A of the fixing part 112 and a center 78B of the distal end-side opening portion 78A. Accordingly, a state where the light guide 28B is bent in the light guide-insertion space 70 is maintained in the rotation range of the fixing member 110. As a result, the torsion of the light guide 28B does not occur in the rotation range of the fixing member 110.

Further, as shown in FIGS. 9 to 11, the connecting beam 100 disposed in the light guide-insertion space 70 together with the light guide 28B is formed in the shape of a beam extending in the direction of the insertion axis Ax in the light guide-insertion space 70. Accordingly, a ratio (space) occupied by the connecting beam 100 in the light guide-insertion space 70 can be kept small. As a result, the light guide 28B can change the posture thereof without being obstructed by the connecting beam 100 in a state where the light guide 28B is bent. It is preferable that the surface of the connecting beam 100 is formed in the shape of a round surface. Accordingly, damage to the light guide 28B in a case where the light guide 28B is in contact with the connecting beam 100 can be prevented.

Here, “a state where tension is not applied” means that additional tension (maximum tension) to be applied to the light guide 28B depending on the rotational operation of the fixing member 110 is substantially 0 (zero). That is, in a case where initial tension generated in the light guide 28B before the rotational operation of the fixing member 110 (including tension depending on the own weight of the light guide 28B) is denoted by TO and additional tension applied to the light guide 28B after the rotational operation of the fixing member 110 is denoted by T1, total tension T0+T1 acts on the light guide 28B after the rotational operation of the fixing member 110. In the oblique-viewing endoscope 10 according to the embodiment, the light guide 28B is housed in the light guide-insertion space 70 in a state where the light guide 28B is bent and the additional tension T1 applied to the light guide 28B after the rotational operation of the fixing member 110 is substantially 0. For this reason, total tension T0+T1 generated in the light guide 28B before and after the rotational operation of the fixing member 110 is substantially constant, so that excessive tension is not generated in the light guide 28B. The additional tension T1 applied to the light guide 28B after the rotational operation of the fixing member 110 is not necessarily limited to 0 (zero) and may be additional tension T1 having a magnitude large enough not to induce the co-rotation of the external cable 72.

Further, in the oblique-viewing endoscope 10 according to the embodiment, the light guide 28B is housed in the light guide-insertion space 70 in a state where the light guide 28B is bent, and an excessive torsional force does not act on the light guide 28B during the rotational operation of the fixing member 110 in the rotation range of the fixing member 110. That is, even in a case where the rotational operation of the fixing member 110 is performed, large tension (total tension) and a large torsional force do not act on the light guide 28B. Accordingly, the breakage (cutting or the like) of the light guide 28B can be prevented.

An aspect in which the light guide 28B is inserted and disposed in a state where the light guide 28B is bent in a wave shape has been described by way of example in this embodiment as one aspect in which tension is not applied to the light guide 28B in the light guide-insertion space 70, but an aspect in which the light guide 28B is inserted into and disposed in the light guide-insertion space 70 while being wound in a loop shape is also conceivable. However, since the light guide 28 is a generally firm member, there is a problem in that excessive tension is applied to the light guide 28B and the light guide 28B is broken in a case where the light guide 28B is disposed while being wound in a loop shape. This problem can be solved in a case where the diameter of a loop is increased to reduce tension. However, since the operation unit body 22 is increased in size in this case, it is not preferable that the diameter of a loop is increased. From such a point of view, it is preferable that the light guide 28B is inserted into and disposed in the light guide-insertion space 70 in a state where the light guide 28B is bent in a wave shape. Accordingly, the breakage of the light guide 28B can be prevented, and the operation unit body 22 can be reduced in diameter.

Further, in the oblique-viewing endoscope 10 according to this embodiment, as shown in FIG. 3, the fixing part 112 of the fixing member 110 is disposed on the distal end side of a proximal end surface 96a of the bearing receiving member 96 in the direction of the insertion axis Ax. Accordingly, the length of the light guide 28B in the light guide-insertion space 70 can be set to be longer than the length of the second signal cable 27. As a result, stress applied to the light guide 28B during the rotation of the fixing member 110 can be suppressed. As another aspect, the fixing part 112 may be disposed on the distal end side of the insertion hole 104a (see FIG. 5), which is formed in the second magnet 104, in the direction of the insertion axis Ax in the case of an aspect in which the second magnet 104 and the connecting beam 100 are connected to each other without the use of the bearing receiving member 96. As a result, the same effects as described above are obtained.

The fixing member 110 shown in FIGS. 9 to 11 is rotated integrally with the case 74 in a case where the knob 36 is operated to rotate. However, since the light guide 28B is entangled in the connecting beam 100 in a case where the rotation of the fixing member 110 is made free (infinite), it is not preferable that the rotation of the fixing member 110 is made free. Accordingly, the oblique-viewing endoscope 10 according to this embodiment comprises a rotation stopper 120 (see FIG. 3) that regulates the rotation range of the fixing member 110 in the direction B around the axis.

FIG. 3 shows an example of the rotation stopper 120. As shown in FIG. 3, the rotation stopper 120 includes a stopper groove 122 that is formed on the operation unit body 22 and a stopper pin 124 that protrudes from the knob 36. The rotation stopper 120 is an example of a rotation stopper of the present invention.

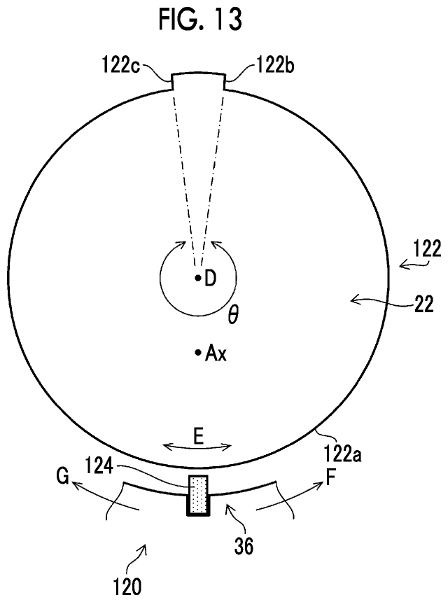

FIG. 13 is a schematic diagram showing the configuration of the rotation stopper 120 in a case where the knob 36 is viewed from the proximal end side of the operation unit body 22. As shown in FIG. 13, the stopper groove 122 is formed on the outer peripheral surface of the distal end portion of the operation unit body 22. The stopper groove 122 includes a groove portion 122a, a wall portion 122b that is formed on one end side of the groove portion 122a, and a wall portion 122c that is formed on the other end side of the groove portion 122a. The groove portion 122a is formed in an arc shape centered on a central axis D of rotation of the knob 36 with respect to the operation unit body 22 on a plane perpendicular to the insertion axis Ax. Further, each of the wall portions 122b and 122c are formed as a stopper surface that protrudes from the groove portion 122a in a normal direction. On the other hand, the stopper pin 124 protrudes from the inner peripheral surface of the knob 36 toward the central axis D of rotation, and is inserted into the groove portion 122a.

Hereinafter, an example of the rotation range of the fixing member 110 regulated by the rotation stopper 120 will be described while a positional relationship between the rotation stopper 120 (stopper pin 124) shown in FIG. 13 and the fixing member 110 (fixing part 112) shown in FIGS. 9 to 11 is described.

In a case where the fixing part 112 is positioned at the middle position in the rotation range of the fixing member 110 as shown in FIG. 9, the stopper pin 124 is positioned at a middle position in a length direction E of the groove portion 122a as shown by a solid line in FIG. 13. After that, in a case where the knob 36 is operated to rotate in a counterclockwise direction F, the stopper pin 124 is moved along the groove portion 122a in the same direction, and the fixing part 112 is rotated in the counterclockwise direction from a position shown in FIG. 9 toward a position shown in FIG. 10. Then, in a case where the stopper pin 124 comes into contact with the wall portion 122b, the fixing part 112 is stopped at the position shown in FIG. 10. Accordingly, the rotation of the fixing member 110 in the counterclockwise direction is regulated. After that, in a case where the knob 36 is operated to rotate in a clockwise direction G from this state, the stopper pin 124 is moved along the groove portion 122a in the same direction, and the fixing part 112 is rotated in the clockwise direction from the position shown in FIG. 10 toward a position shown in FIG. 11. Then, in a case where the stopper pin 124 comes into contact with the wall portion 122c, the fixing part 112 is stopped at the position shown in FIG. 11. Accordingly, the rotation of the fixing member 110 in the clockwise direction is regulated. As described above, the rotation range of the fixing member 110 is regulated (defined) by the rotation stopper 120. Since the rotation range of the fixing member 110 is regulated in this way, a problem in that the light guide 28B is entangled in the connecting beam 100 can be solved.

In FIG. 13, the rotation range of the fixing member 110 regulated by the rotation stopper 120 is represented by an angle θ. In terms of preventing the entanglement, the angle θ is preferably at least 350° or less and may be 300° or less or 200° or less. Further, the angle θ may be set depending on the type of an oblique-viewing endoscope.

A configuration in which the stopper groove 122 is formed on the operation unit body 22 and the stopper pin 124 protrudes from the knob 36 has been described as the rotation stopper 120 by way of example in this embodiment, but any configuration can be applied as long as the rotation range of the fixing member 110 can be regulated. For example, a configuration in which a stopper groove 122 is formed on the inner peripheral surface of the operation unit body 22 and a stopper pin 124 protrudes from the outer peripheral surface of the case 74 can also be applied as the rotation stopper 120.

Meanwhile, the light guide 28B has a length that allows a bent state to be maintained between the fixing part 112 and the distal end-side opening portion 78A in the direction of the insertion axis Ax in the rotation range (angle θ) of the fixing member 110. For example, it is preferable that the length of the light guide 28B present between the fixing part 112 and the distal end-side opening portion 78A is a length of 1.2 to 1.5 times the linear distance L between the proximal end 112A of the fixing part 112 and the center 78B of the distal end-side opening portion 78A as shown in FIG. 12. Accordingly, a state where tension is not applied to the light guide 28B and the light guide 28B is bent in a wave shape in the light guide-insertion space 70 without being bent in a loop shape is maintained in the rotation range (angle θ) of the fixing member 110.

Next, the action of the oblique-viewing endoscope 10 according to the embodiment will be described.

In the oblique-viewing endoscope 10 according to the embodiment, a practitioner grips the operation unit body 22 and inserts the insertion unit 20 into a patient's body and then rotationally operates the knob 36 in the direction B around the axis in a case where a visual field direction is to be changed. Then, the outer pipe 30 and the protection sheath 32 to be rotated integrally with the knob 36 are rotated in the same direction, and the visual field direction can be directed to a desired direction. Further, in a case where the practitioner rotationally operates the outer pipe 30 using the knob 36, the rotation (co-rotation) of the protection sheath 32 and the inner sheath 34 (the proximal end optical system 50 and the image pickup unit 60) in the direction B around the axis is prevented. That is, since the posture of the inner sheath 34 in the direction B around the axis is maintained by the magnet coupling 102, the rotation of an observation image to be observed on the monitor 16 is prevented even though the visual field direction is changed. As a result, the operability of the oblique-viewing endoscope 10 is improved.

In addition, in the oblique-viewing endoscope 10 according to the embodiment, the light guide 28A provided on the distal end side of the fixing part 112 is rotated integrally with the outer pipe 30 and the protection sheath 32 together with the fixing member 110 (fixing part 112) in a case where the knob 36 is operated to rotate. However, the bent state of the light guide 28B provided on the proximal end side of the fixing part 112 is maintained in the light guide-insertion space 70 even though the fixing member 110 is rotated. Accordingly, a state where the light guide 28B is inserted into the external cable 72 from the distal end-side opening portion 78A without torsion is maintained. As a result, since the practitioner does not receive a reaction force caused by torsion from the light guide 28B and the external cable 72 in a case where the practitioner gripping the operation unit body 22 rotates the outer pipe 30, it is easy to perform an operation for changing a visual field direction.

As described above, the oblique-viewing endoscope 10 according to the embodiment employs a configuration in which the fixing member 110 to be rotated integrally with the knob 36 in the direction B around the axis is disposed in the operation unit body 22, a part of the light guide 28 in the longitudinal direction is fixed by the fixing part 112 of the fixing member 110, the light guide-insertion space 70 is formed in the operation unit body 22, and the light guide 28B is inserted into and disposed in the light guide-insertion space 70 so that tension is not applied to the light guide 28B present between the fixing part 112 and the distal end-side opening portion 78A even in a case where the fixing member 110 is rotated in the direction B around the axis by the knob 36. Accordingly, the structure of the oblique-viewing endoscope 10 is simple, and the co-rotation of the external cable 72 can be prevented.

Further, in the oblique-viewing endoscope 10 according to the embodiment, since a reaction force caused by the torsion of the light guide 28 and the external cable 72 is not applied to the operation unit body 22, it is easy to hold the operation unit body 22 at an optimum position in the observation direction. As a result, the operability of the oblique-viewing endoscope 10 is significantly improved.

### Other Embodiments

The multicore cable including the plurality of strands (signal lines), the shield conductor, and the sheath has been described as the first signal cable 26 and the second signal cable 27 by way of example in the above-mentioned embodiment, but the first signal cable 26 and the second signal cable 27 are not limited thereto. For example, as another embodiment, the first signal cable 26 and the second signal cable 27 can also be formed of a plurality of separated strands. Then, even if a force in a torsional direction (torque) acts on the first signal cable 26 and the second signal cable 27, the torque can be reduced. Accordingly, the disconnection of the first signal cable 26 and the second signal cable 27 can be prevented. Further, in a case where the second signal cable 27 is formed of the above-mentioned strands, the strands may be in contact with the peripheral edge portion of the distal end-side opening portion 78A shown in FIG. 3. However, since the peripheral edge portion of the distal end-side opening portion 78A is formed in the shape of a round surface, there is an advantage in that the disconnection of the strands can be prevented.

Other

The annular knob 36 has been described as the rotational operation member by way of example in the above-mentioned embodiment. However, for example, a configuration in which a member to be easily accessible to practitioner's fingers, such as a convex member or a serrated member, is formed on a part of the outer peripheral surface of the outer pipe 30 can also be applied.

Examples of the endoscope according to the embodiment of the present invention have been described above, but the present invention may include some improvements or modifications without departing from the scope of the present invention.

### EXPLANATION OF REFERENCES

10: oblique-viewing endoscope
12: endoscope system
14: processor device
16: monitor
18: light source device
20: insertion unit
22: operation unit body
24: camera unit
26: first signal cable
27: second signal cable
28: light guide
28A: light guide
28B: light guide
28C: light emitting end
30: outer pipe
31: space
32: protection sheath
34: inner sheath
36: knob
38: seal ring
40: distal end optical system
42: distal end portion body
44: distal end lens barrel
45: tubular portion
46: cover glass
48a: objective lens
48b: prism
48c: lens
50: proximal end optical system
52: proximal end lens barrel
54: holder
55: prism
56: lens
60: image pickup unit
64: image pickup element
66: circuit board
68: connector
70: light guide-insertion space
72: external cable
74: case
76: cable body
78: connection tube
78A: distal end-side opening portion
77: metal fitting
79: O-ring
80: sealed space
82: air-tight connector
84: connecting unit
90: connection member
92: bearing receiving member
94: bearing
96: bearing receiving member
96a: proximal end surface
98: bearing
100: connecting beam
100a: ring portion
100b: ring portion
102: magnet coupling
103: first magnet
103a: insertion hole
104: second magnet
104a: insertion hole
110: fixing member
111: flange
112: fixing part
113: screw
120: rotation stopper
122: stopper groove
122a: groove portion
122b: wall portion
122c: wall portion
124: stopper pin
Ax: insertion axis
OA: optical axis
B: direction around axis
C: central axis
D: central axis of rotation
E: length direction
F: counterclockwise direction
G: clockwise direction
L: linear distance

What is claimed is:

1. An endoscope comprising:
an outer pipe that forms an insertion unit;
a pipe-like operation unit body that is connected to a proximal end side of the outer pipe and that supports the outer pipe to allow the outer pipe to be rotatable in a direction around an axis of the insertion unit;
an annular rotational operation member that is fixed to the proximal end side of the outer pipe and that rotates the outer pipe in the direction around the axis with respect to the operation unit body;
a protection sheath that is inserted into the outer pipe and that is rotatable integrally with the outer pipe;
a distal end optical system that is provided on a distal end side of the protection sheath;
a flexible light guide that is disposed in a space between the outer pipe and the protection sheath and that includes a light emitting end on a distal end side of the outer pipe;
an inner sheath that is inserted into the protection sheath and that is rotatable relative to the protection sheath in the direction around the axis;
an image pickup unit that is provided on a distal end side of the inner sheath and that picks up an image of light passing through the distal end optical system;
an external cable which is connected to a proximal end side of the operation unit body and into which the light guide is inserted from a distal end-side opening portion; and
a fixing member that is disposed in the operation unit body, includes a fixing part for fixing a part of the light guide in a longitudinal direction, and is rotatable integrally with the rotational operation member in the direction around the axis,
wherein a light guide-insertion space is formed between the fixing part and the distal end-side opening portion in the operation unit body, and
in a case where the fixing member is rotated in the direction around the axis by the rotational operation member, the light guide is inserted into and disposed in the light guide-insertion space in a state where tension is not applied to the light guide present between the fixing part and the distal end-side opening portion.

2. The endoscope according to claim 1,
wherein the fixing member is connected to a proximal end side of the protection sheath and is rotatable integrally with the protection sheath around the axis.

3. The endoscope according to claim 2,
wherein the fixing member is formed of a ring-shaped member connected to the proximal end side of the protection sheath and includes the fixing part at a part of the ring-shaped member in a circumferential direction.

4. The endoscope according to claim 1, further comprising:
a rotation stopper that regulates a rotation range of the fixing member around the axis.

5. The endoscope according to claim 4,
wherein the light guide has a length that allows a bent state to be maintained between the fixing part and the distal end-side opening portion in an axial direction of the insertion unit in the rotation range regulated by the rotation stopper.

6. The endoscope according to claim 1,
wherein the rotational operation member is provided on a distal end side of the operation unit body and is formed of an annular member rotatable in the direction around the axis with respect to the operation unit body.

7. The endoscope according to claim 1, further comprising:
a signal cable that is connected to the image pickup unit and that is inserted into the inner sheath,
wherein the signal cable is inserted into the external cable from the distal end-side opening portion.

8. The endoscope according to claim 7,
wherein the signal cable is a plurality of separated strands.

9. The endoscope according to claim 1, further comprising:
a pipe-like case that is connected to a proximal end side of the protection sheath in the operation unit body and that is disposed on a distal end side of the light guide-insertion space in an axial direction of the insertion unit;
a partition wall that is provided in the case and that is perpendicular to an insertion axis of the insertion unit; and
a magnet coupling which includes a first magnet provided on a distal end side in the axial direction and a second magnet provided on a proximal end side in the axial direction with the partition wall interposed therebetween and of which the first magnet is connected to a proximal end side of the inner sheath,
wherein the magnet coupling and the case are rotatable relative to each other in the direction around the axis.

10. The endoscope according to claim 9,
wherein a distal end side of the external cable is connected to a proximal end side of the second magnet via a connecting member formed in a shape of a beam extending in the axial direction in the light guide-insertion space.

11. The endoscope according to claim 9, further comprising:
a signal cable that is connected to the image pickup unit and that is inserted into the inner sheath,
wherein each of the first magnet and the second magnet is formed in a shape of a disk perpendicular to the insertion axis and includes an insertion hole into which the signal cable is to be inserted.

12. The endoscope according to claim 11,
wherein the fixing member is fixed to an outer peripheral surface of the case, and
the fixing part of the fixing member is disposed on a distal end side of the insertion hole, which is formed in the second magnet, in the axial direction.

13. The endoscope according to claim 1,
wherein a peripheral edge portion of the distal end-side opening portion of the external cable is formed in a shape of a round surface.

14. The endoscope according to claim 1, further comprising:
a proximal end optical system that is provided on the distal end side of the inner sheath and that guides light, which passes through the distal end optical system, to the image pickup unit,
wherein the image pickup unit includes an image pickup element that picks up an image of light, which is incident through the proximal end optical system, and that outputs an image pickup signal to a signal cable, and
the distal end optical system is rotatable in the direction around the axis with respect to the proximal end optical system and the image pickup element.

15. An endoscope comprising:
an outer pipe that forms an insertion unit;
a pipe-like operation unit body that is connected to a proximal end side of the outer pipe and that supports the outer pipe to allow the outer pipe to be rotatable in a direction around an axis of the insertion unit;
an annular rotational operation member that is fixed to the proximal end side of the outer pipe and that rotates the outer pipe in the direction around the axis with respect to the operation unit body;
a protection sheath that is inserted into the outer pipe and that is rotatable integrally with the outer pipe;
a distal end optical system that is provided on a distal end side of the protection sheath;
a flexible light guide that is disposed in a space between the outer pipe and the protection sheath and that includes a light emitting end on a distal end side of the outer pipe;
an inner sheath that is inserted into the protection sheath and that is rotatable relative to the protection sheath in the direction around the axis;
an image pickup unit that is provided on a distal end side of the inner sheath and that picks up an image of light passing through the distal end optical system;
an external cable which is connected to a proximal end side of the operation unit body and into which the light guide is inserted from a distal end-side opening portion; and
a fixing member that is disposed in the operation unit body, includes a fixing part for fixing a part of the light guide in a longitudinal direction, and is rotatable integrally with the rotational operation member in the direction around the axis,
wherein a light guide-insertion space is formed between the fixing part and the distal end-side opening portion in the operation unit body, and
the light guide inserted into and disposed in the light guide-insertion space has a length longer than a linear distance between a proximal end of the fixing part and a center of the distal end-side opening portion.

16. The endoscope according to claim 15,
wherein the fixing member is connected to a proximal end side of the protection sheath and is rotatable integrally with the protection sheath around the axis.

17. The endoscope according to claim 15, further comprising:
a rotation stopper that regulates a rotation range of the fixing member around the axis.

18. The endoscope according to claim 15,
wherein the rotational operation member is provided on a distal end side of the operation unit body and is formed of an annular member rotatable in the direction around the axis with respect to the operation unit body.

19. The endoscope according to claim 15, further comprising:
a signal cable that is connected to the image pickup unit and that is inserted into the inner sheath,
wherein the signal cable is inserted into the external cable from the distal end-side opening portion.

20. The endoscope according to claim 15, further comprising:
a pipe-like case that is connected to a proximal end side of the protection sheath in the operation unit body and that is disposed on a distal end side of the light guide-insertion space in an axial direction of the insertion unit;
a partition wall that is provided in the case and that is perpendicular to an insertion axis of the insertion unit; and
a magnet coupling which includes a first magnet provided on a distal end side in the axial direction and a second magnet provided on a proximal end side in the axial direction with the partition wall interposed therebetween and of which the first magnet is connected to a proximal end side of the inner sheath,
wherein the magnet coupling and the case are rotatable relative to each other in the direction around the axis.
